(19) United States
(12) Patent Application Publication
(10) Pub. No.: US 2009/0217931 A1
(43) Pub. Date: Sep. 3, 2009

(54) Tongue-Holding Device

(75) Inventors: Graeme Howard Davies, London (GB); Christopher Huntley, London (GB); Lisa Maria Stroux, London (GB); Philip Ronald Greer, London (GB)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: Royal College of Art, London (GB)

(21) Appl. No.: 12/278,752

(22) PCT Filed: Feb. 9, 2007
(86) PCT No.: PCT/GB2007/000457

§ 371 Date: Nov. 10, 2008

(30) Foreign Application Priority Data
Feb. 10, 2006 (EP) 06250746.2

(51) Int. Cl. A61F 5/37

(52) U.S. Cl. 128/860

(57) ABSTRACT

The present invention provides a device for holding a tongue to hold a person's airway open, for example when a person is unconscious. The device comprises a chamber configured to snugly accommodate a tongue, and a resilient hollow bulb that is in fluid communication with the chamber. The chamber has an open end through which a person's tongue can project. The device is configured such that the deforming and releasing of the bulb creates a reduced pressure in the chamber, thereby retaining the tongue within the chamber. The device prevents the tongue from blocking the airway.

### TECHNICAL FIELD

This invention relates to a non-invasive device for holding open the airway of a person, for example because they are unconscious.

### BACKGROUND ART

When emergency services paramedics arrive on the scene of an incident, the first action they take is to check whether an unconscious casualty is breathing. If a casualty is not breathing, the paramedics will insert an airway tube into the patient's mouth to prevent the patient suffocating on their own tongue. However, this action often comes too late, since, to give an unconscious patient the best chance of survival, their airway must be cleared within 4 minutes.

The device currently used by the emergency services is a plastic tube, which is curved at the end to be inserted into a patient's throat. This type of tube can be difficult to insert and fit correctly, hence training in performing this procedure is given to emergency services workers. The tube is inserted upside down and rotated 180 degrees to fit behind the tongue. The tube comes in seven different sizes, and all seven must be carried as a set to accommodate as wide as possible a spectrum of casualties.

The currently used device gives no accurate feedback on whether it has been inserted correctly, or whether the correct size for the patient has been chosen, hence it is not obvious whether it is working properly. Further, if the device is wrongly inserted, or if the wrong size is used, it could potentially injure the patient's throat. For example the tip of the tube may interfere with the epiglottis, exacerbating the problem, or might stimulate the Vagus nerve at the back of the throat, stopping the heart. If the patient is not fully unconscious, the gag reflex may induce vomiting, which may cause further blockage of the airway.

First Aid courses teach participants how to manually clear the airway of an unconscious patient, and this can be done by administering a jaw-thrust, jaw-lift or tongue pull. This type of airway clearance may particularly be necessary where a first-aider is unable to move the injured person, for example in the case of a spinal injury, or a suspected spinal injury, or where the casualty is trapped, for example by machinery or other debris. However, few members of the public attend such courses and this method also requires the first-aider to maintain a precise position for the period of time for which the patient is unconscious, which could be lengthy and uncomfortable. This need to stay with the patient when manually ensuring that the airway remains clear, also prevents a first-aider from performing any other necessary first-aid on the patient, such as attending to a bleeding wound, or from helping any other casualty at the scene that may also be in need of first aid, or for seeking additional assistance, and for preventing further causes of injury.

U.S. Pat. No. 5,373,859 describes a tongue holding device with a housing that tapers outwardly from an opening, and a squeezable vacuum device. The device holds the tip of a user's tongue and can be used to control snoring, by preventing the tongue from blocking the airway, and for unconscious patients. The device may further have a shield which holds the device against the user's face during use.

U.S. Pat. No. 4,196,724 describes a tongue holding device having a housing which tapers inwardly from an opening to a chamber for holding the tip of a person's tongue. The tip of the user's tongue is wedged into the chamber. A vacuum bulb can be used to draw the tip of the tongue into the chamber and to maintain a reduced pressure within the chamber.

US 2002/0139375 describes a tongue-holding device for treating sleep disorders. The device has a mouthpiece with a chamber to hold the forward part of the tongue. In use, the mouthpiece rests against the user's teeth. The chamber can be evacuated via a suction channel connecting the mouthpiece to a suction source.

WO 01/43673 describes an anti-snoring device having a body with an opening leading to a hollow interior that holds the user's tongue, and a bulb to create a negative pressure within the interior to hold the tongue. The device is made of a resiliently flexible material, and is provided with tabs that engage the user's face to hold the device and hence the tongue forward, thereby assisting in keeping the airway open and reducing snoring.

U.S. Pat. No. 5,465,734 describes an anti-snoring device that holds a user's tongue forward to decrease airway obstruction and thereby reduce snoring. The device has a tongue holding chamber of an hourglass shape. This shape provides mechanical retention of the tongue. A vacuum can be used to pull the tongue into and/or retain the tongue in the chamber. A shield is provided to engage the user's lips to hold the tongue forward.

### DISCLOSURE OF THE INVENTION

The invention is set out in the claims. In general terms it provides a device for holding a tongue to hold a person's airway open and comprises a chamber configured to snugly accommodate a tongue and a resilient, hollow bulb in fluid communication with the chamber. The chamber has an open end through which a person's tongue can project into the chamber; the device is configured such that the deforming and releasing of the bulb creates a reduced pressure (i.e. a pressure below atmospheric pressure) in the chamber thereby retaining a person's tongue within the chamber. Preferably the chamber tapers from its open end towards the bulb, to assist it in fitting snugly around a patient's tongue.

The chamber is preferably approximately ‘tongue shaped’, which allows for a large surface contact area between the walls of the chamber and the patient's tongue, and helps spread the suction force across the tongue, reducing the effects of ischemia. Further, this shape assists the formation of a strong seal between the tongue and the chamber walls, and reduces the risk of the device coming loose in use. The tapered shape of the chamber wall from the open end of the chamber towards the bulb enables the chamber to fit both large and small tongues.

The open end of the chamber preferably has a shaped edge to give the best fit to a tongue. The edge is preferably made of soft rubber to maximise patient comfort. The chamber wall may also be shaped to give a good fit inside the mouth, and to facilitate application to a tongue.

The patient has to be able to breathe when the device is fitted and so an air passageway must be provided that allows air to enter the patient's mouth despite the presence of the device. Such a passageway could be provided within the device for this purpose, e.g. a passageway provided on the outer wall of the chamber or the device could be so shaped that the patient's lips do not form a seal around the whole periphery of the device, thereby forming the passageway between the patient's lips and the device. Alternatively a separate tube may be provided to form the passage allowing breathing air to enter into the patient's mouth. In one embodiment, an indentation may be provided along one of the top or bottom surfaces of the chamber wall that prevents the patient's lips forming a seal around the device and thereby allowing air to pass into the mouth of the patient.

The chamber wall is preferably transparent, allowing the patient's tongue to be viewed through the wall when it is inside the chamber so that it can be seen from the outside that the device is fitted properly. The wall is preferably rigid, thereby acting as a bite guard and protecting the tongue and preventing it from being bitten, e.g. in the case of the patient having a fit.

The bulb is compressible and so can be evacuated when deformed and is resilient so that it will maintain a suction force on the chamber. In order to create a sufficiently powerful suction in the tongue chamber, the internal volume of the bulb is preferably at least equal to the internal volume of the chamber, so that even incomplete compression and evacuation of the bulb will enable the device to work. Preferably, the internal volume of the bulb is greater than the volume of the chamber. Due to the suction force created, no mechanism for locking or holding the device in place is necessary.

The bulb is in fluid communication with the chamber so that the reduced pressure in the bulb is transmitted to the chamber. Many different forms of connection are possible. For example, the device may include a specific connector, e.g. a section connecting the chamber to the bulb. As the chamber wall tapers towards the bulb, the chamber wall may form a channel extending into the bulb. This channel provides a leak-tight connection between the bulb and the chamber. The chamber wall surrounding the channel may have a greater wall thickness than the remaining chamber wall to assist in preventing the channel between the chamber and bulb from being closed inadvertently, for example if the patient bites on the connector during a fit. If the channel were to be closed, the reduced pressure in the chamber could rise, and the tongue holder could be released.

The device is simply operated as follows: As the bulb is compressed, air is pushed out of the bulb. The tongue of the patient is inserted into the chamber and the bulb is released to form a reduced pressure inside the bulb. The tongue is drawn into the chamber by the suction force created by the bulb, and a tight seal is formed between the tongue and the internal wall of the chamber, enabling the device to stay fixed on the tongue. The tongue may be inserted into the chamber before the bulb is compressed and a valve may be provided to vent the air expelled from the bulb.

The seal between the tongue and the tapered shape of the chamber wall from the open end of the chamber towards the bulb, is assisted, but not reliant upon, the saliva on the tongue.

The shape of the device is preferably such to hold open the patient's airway, by preventing the tongue from sliding back into the throat.

The device of the present invention provides three mechanisms to prevent the tongue from falling to the back of the throat:

(1) The size and shape of the bulb is such that it is difficult for the device to fall back into the mouth.
(2) The lower lip of the chamber acts against the Frenulum Lingua preventing the device from slipping further into the mouth.
(3) The weight of the bulb and chamber can act as anchorage, pulling the tongue out of the mouth to one side.

As explained above, the device can be used on an unconscious person to retrieve the tongue from the back of the mouth and draw it forward out of the throat to prevent obstruction of, or to clear, the airway. This may be done by a person, for example a person giving first aid. The person compresses the bulb and inserts the device into the mouth of an unconscious person. The length of the device enables it to reach right to the back of the mouth. When the opening of the device is placed in the vicinity of the tongue, the bulb can be fully or partially released in order to pull the tongue into the chamber.

The dimensions of the device should be such that the device can reach the back of the mouth. This ‘fishing’ for the tongue is facilitated by chamber walls of the device being rigid. The chamber walls are transparent to allow a person using the device to see when the tongue is engaged in the chamber of the device. Additionally, the device of the present invention need not have a shield or tab to engage with the user's face or teeth to prevent the device being drawn to the back of the throat since the device can be sufficiently weighted that it acts as an anchorage to hold the tongue out of the mouth. The absence of a shield or tabs also allows a person giving first aid to have an unobstructed view when fitting the device. For the same reason, the bulb is preferably of an oval shape. The oval shape also allows the flattened bulb and the fingers flattening it to reach as far as necessary into the mouth and to be able to manoeuvre the device conveniently. Thus a person using the device is able to either reach into the mouth with his fingers, as well as the device, or to avoid reaching his fingers into the mouth of an unconscious person by merely using the device to reach for and engage the tongue in the device.

The device of the present invention provides several advantages over the type(s) of device(s) currently in use:
One size of device can work on a wide range of different tongue sizes, thanks to the compliance of the tongue. If the device is used on a very large tongue, the difference will be only that the tongue would not project/be drawn as far into to chamber as a smaller tongue. Therefore, only one size of device needs to be carried or stocked, rather than several different sizes. This also relieves the first-aider from having to make a decision under pressure regarding which size of device is most suitable for the patient, saving valuable time.
The device of the present invention is very easy to use and requires no prior training. It may be used, both by professional first-aiders and untrained bystanders attending a casualty.
In use, this device does not extend into the throat, thereby overcoming any problems of injuring the back of the throat by incorrect insertion or by using an inappropriate size of a currently used airway device. The device is located only in the front of the mouth, making it easy to handle and safer to use.
When the chamber wall is transparent or translucent, the device can allow immediate feedback that the device has been correctly applied and is working correctly, since, if the device is attached to the tongue, it is working. Further, one is able to see whether the device is working from a distance by inspecting the state of the bulb.
If the walls of the chamber are rigid, the device protects the patient from injury for example by biting his own tongue during a fit.
The internal surface of the chamber wall may comprise carefully designed ‘non-slip’ surface contact areas to ensure that an optimal seal is created between the tongue and the chamber wall, preventing any loss of suction.
Very little contact with the patient is necessary when applying the device, which agrees with the present trend in first aid to minimise personal contact with the patient. The device is designed such that the first-aider need not penetrate the oral cavity of the casualty with their fingers. The device also complies with the current teaching to first-aiders to move away from mouth-to-mouth resuscitation and to focus more on chest compressions.
Use of the device of the present invention allows first-aiders to treat several patients, as once the device has been fitted, the airway can be left unsupervised for extended periods.
The device reduces the need to move a casualty into the recovery position. This is particularly important where the casualty may have a spinal injury, where repositioning them could result in paralysis.
The device is small and is easily fitted into a first aid box, and it is possible for a significant number to be carried in ambulance vehicles. Its shape and colour can make it easily identifiable.
The device can be mass produced at low cost.
The device may act as a form of triage, as it protrudes from the mouth of the patient, thereby allowing arriving paramedics to quickly and easily see which patients have been treated. The use of a brightly coloured material for the bulb section is also helpful in this regard.

### BRIEF DESCRIPTION OF THE FIGURES

A specific embodiment of the invention will now be described by way of example with reference to the accompanying drawings:

FIG. 1 is a perspective view of the device of the present invention

FIG. 2 is a vertical schematic view of the device showing the textured grip pattern on one side of the bulb

FIG. 3 is a vertical schematic view of the device showing the textured grip pattern on opposing sides of the bulb

FIG. 4 is a schematic top view of the device

FIG. 5 is a vertical cross-sectional view of the device (A-A on FIG. 2).

FIG. 6 is a partial cross-sectional view of the snap fit part of the device connecting the bulb to the chamber wall (detail B on FIG. 5).

### DETAILED DESCRIPTION AND BEST MODE FOR CARRYING OUT THE INVENTION

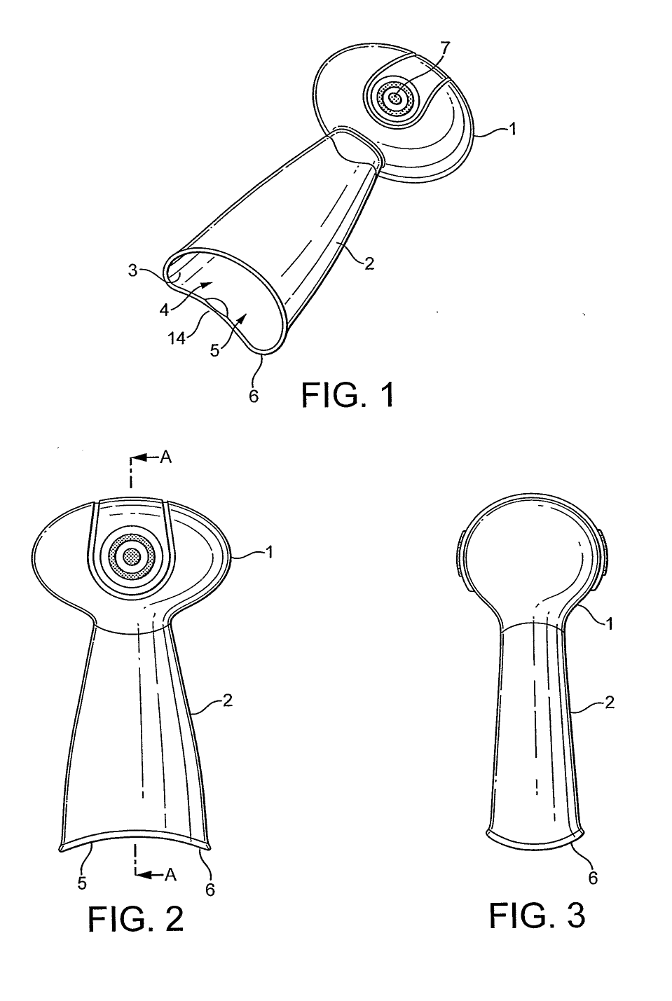

Referring initially to FIG. 1, there can be seen a tongue-holding device in accordance with the present invention.

The tongue holding device shown in FIG. 1 includes a bulb 1 connected to a chamber wall 2. The internal surface 3 of the chamber wall defines a chamber 4. The chamber has an open end 5, the edge 6 of the open end being a soft rubber lip. The chamber wall tapers from the open end towards the bulb. The bulb has a textured section 7 for ease of use, increased grip and communication of operation. (see FIGS. 2 and 4).

The chamber wall is preferably approximately ‘tongue-shaped’ to give the most comfortable and snug fit to a tongue. The chamber is therefore of an approximately elliptical cross-sectional shape. Additionally, the upper and lower parts of the chamber may be of different shapes to fit the upper and underneath sides of the tongue. The chamber wall may also be shaped to provide an indentation 14 along one of the top or bottom surfaces of the chamber wall, to allow air to pass into the mouth of the patient. The bulb is spherically shaped, but other shapes are possible.

FIGS. 2 and 3 show side views of the device. It can be seen that the edge 6 of the open end of the chamber wall 2 at the open end of the chamber 5 may be shaped in order to accommodate the Frenulum Lingua, which is the stretched band of skin between the tongue and the base of the mouth of the patient. The tapering of the chamber wall 2 from the open end of the chamber towards the bulb 1 to snugly accommodate a tongue is clearly illustrated in these figures. The chamber wall may also be shaped to provide an indentation 14 along one of the top or bottom surfaces of the chamber wall, thereby allowing air to pass into the mouth of the patient.

FIG. 4 shows a top view of the bulb 1 and the location of textured areas 7 on two opposing sides of the bulb to assist a user in compressing the bulb. When the bulb is not spherical, these textured areas are preferably on the two opposing sides that are closest to each other.

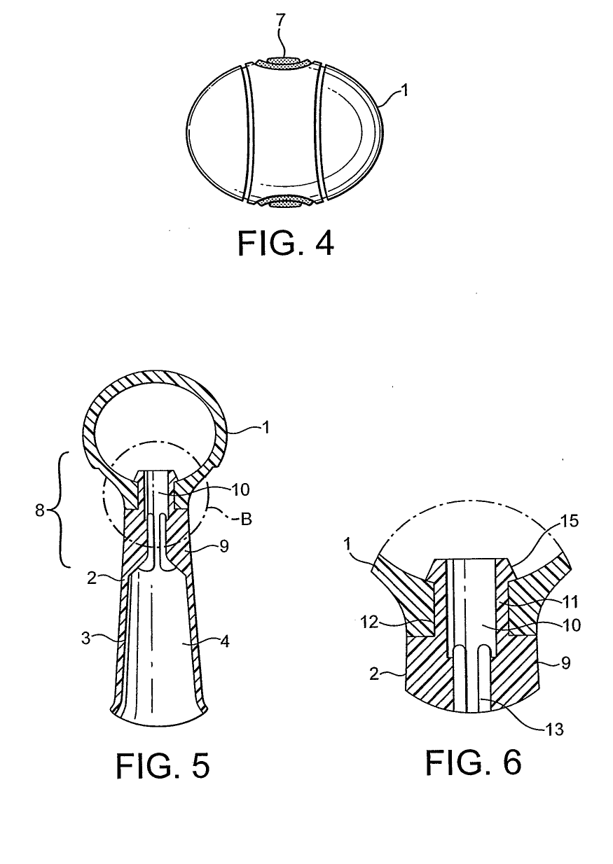

FIG. 5 is an axial cross-sectional view of the device and FIG. 6 is a detail of FIG. 5. The inside surface 3 of the chamber wall tapers from the open end of the chamber towards the bulb; a connector section 8 joins the chamber wall to the bulb. The thickness of the wall 9 of the connector section is greater than the wall thickness of the chamber. The connector section includes an internal channel 10 of a substantially uniform diameter, which extends from the tapered end of the chamber to the inside of bulb 1. The connector section 8 includes a projection 11 that fits into the neck 12 of the bulb 1 and the bulb is held on the projection 11 partly by the resilience of the neck of the bulb and partly by a lip provided at the leading end 15 of the connector section. Material saving ribs 13 are situated inside the channel, to reinforce the channel and to prevent the tongue from being pulled into the channel and bulb.

The wall of the chamber can be made of a tough, rigid material, as this will protect the tongue in case of the patient having a fit. The connector section 8 shown in FIGS. 5 and 6 may be additionally reinforced, thereby ensuring that the force of a patient's bite is not able to close off the channel and thereby inadvertently release the tongue holder. This is achieved by thicker plastic material in this section and can additionally be due to re-enforcing the channel inside the connector.

The total length of the device may be approximately 70 to 120 mm, preferably about 75 to 95 mm, and in one embodiment is about 82.5 mm. The length of the wall of the chamber may be approximately 40 to 80 mm, preferably about 50 to 60 mm. The width of the chamber opening 6 in the narrower direction (corresponding to the chamber wall surfaces that will contact with the upper and lower surfaces of the tongue), is 5 to 20 mm, for example about 12 mm, while the opening in the wider direction (corresponding to the chamber wall surfaces that will contact with the left and right side surfaces of the tongue), has a maximum diameter of about 20 to 60 mm, preferably about 35 to 45 mm. The width of the chamber at its tapered end is approximately 5 to 10 mm, preferably about 8 mm, in the narrower direction, while in the wider direction of its tapered end, the chamber is about 10 to 25 mm wide, preferably about 10 to 15 mm wide.

The thickness of the chamber wall is about 1 to 3 mm, preferably about 2 mm. The wall thickness of the bulb is approximately 1 to 3 mm, preferably about 2 mm. The specific thickness of the walls of the device depends on the material used for its manufacture and should be such as to prevent the patient closing the connection between the bulb 1 and the chamber 2 by biting.

The external width of the device is preferably 37 to 51 mm at its widest point. This range is calculated by taking the preferred diameter of the chamber opening at its widest point, 35 to 45 mm, and adding to this value, the thickness of the chamber wall, i.e. 1 to 3 mm, for both sides of the device.

The external width of the device at the widest point of the chamber opening will therefore preferably be between 37 mm, calculated by taking the 35 mm value and adding 1 mm for each side of the chamber wall to give 37 mm, as the bottom end of the preferred range, and 51 mm, calculated by taking the 45 mm value and adding 3 mm for each side of the chamber wall to give 51 mm as the top end of the preferred range.

The external height of the device is preferably 7 to 26 mm, at its point of maximum height. This range is calculated analogously to the calculation described above, by taking the preferred diameter of the chamber opening at its narrowest point, 5 to 20 mm, and adding to this value, the thickness of the chamber wall, i.e. 1 to 3 mm, for both sides of the device.

Accordingly, the external height of the device at the narrowest point of the chamber opening will preferably be between 7 mm, calculated by taking the 5 mm value and adding 1 mm for each side of the chamber wall to give 7 mm, as the minimum of the preferred range, and 26 mm, calculated by taking the 20 mm value and adding 3 mm for each side of the chamber wall to give 26 mm as the maximum of the preferred range.

The device preferably comprises two parts, viz. the wall of the chamber and the bulb. The chamber wall is preferably manufactured by injection-moulding of a plastic suitable for medical use, for example polypropylene with a high degree of translucency or optical clarity. Various medical grades of polypropylene are commercially available and would be suitable for this application.

The bulb is preferably manufactured by gas-injection-moulding of a thermoplastic elastomer (TPE), although any resilient, flexible plastic, suitable for medical use, would be acceptable. Various medical grades of TPE are commercially available and would be suitable for this application. This method of manufacture allows for a hollow vessel with close tolerances in important locations. In the device of the present invention, the surfaces of the bulb that are in contact with the chamber wall and/or the cylindrical projection 11 of the chamber wall must provide an air-tight seal.

In an alternative embodiment, the device of the present invention could be manufactured in a single co-moulded piece by injection-, or blow moulding. This could for example be achieved by layering TPE over polypropylene, and would additionally allow for a soft lip along the edge of the open end of the chamber. In a further embodiment of the present invention, the device could be manufactured as a single piece, from a single material, by gas-injection moulding. A suitable material is for example a TPE such as DuPont's Hytrel®.

By careful selection of the materials suitable for manufacturing the device, the device could be used in chemically harsh environments, for example petrochemical plants.

The connector section 8 of the chamber wall may additionally be provided with ribs. The ribs could be aligned in parallel to the open end of the chamber and extend from the bulb end of the chamber wall in the direction of the open end of the chamber. The ribs would have a greater wall thickness than the remaining chamber wall thickness, serving as a stronger bite guard and hence further increasing the protection of the tongue. If such ribs are present, the thickness of the chamber wall would be increased to about 3 to 7 mm at the thickest part of a rib.

The device may be coloured, in particular the bulb may be brightly coloured. This may be particularly useful in situations where emergency services have a large number of casualties to attend to, as it would be possible to ascertain quickly, and from a distance, whether the device has already been applied to a casualty to ensure the airway has been cleared and, by looking at the state of inflation of the bulb whether the device has been correctly applied and is working. A bright colour, for example orange or yellow, would be preferable. Alternative or additional visual, auditory, or other sensory means to indicate that the device has been correctly applied to a tongue, may also be incorporated, for example a pressure-operated LED light.

When the patient no longer needs the tongue holder, it can be easily removed by compressing the bulb, thereby raising the pressure inside the chamber to near atmospheric pressure or above, and sliding the tongue out of the device.

The device will generally be disposed of after use, but can be made reusable by sterilisation after use.

The device of the present invention may be sold individually, or supplied as part of a first aid kit for domestic use, or use in cars, businesses or offices, and could additionally be carried by ambulance crew and used in hospitals.

The device of the present invention may be used by epileptics, before, or after a seizure, to prevent the person from biting their tongue, before the initial seizure or fit, or subsequent follow-on seizure or fits.

The device of the present invention may also be used on conscious patients, who may fall in and out of consciousness. The device may also be used in conjunction with chest compressions to treat cardiac related emergencies, for example a heart attack.

The device may be used on either a conscious or an unconscious patient during certain medical procedures. The device could for example be used to hold the tongue out of the way during procedures where an object, such as a probe, or a fibre-optic scope, needs to be passed down the throat, or when the throat needs to be inspected.

The device of the present invention may also be used to prevent Apnoea and obstructed sleep (i.e. snoring). The device of the present invention is superior to many existing devices, as it allows air to pass around the device, rather than closing the mouth area completely.

1. A device for holding a tongue to hold a person's airway open, comprising:
a chamber configured to accommodate a tongue snugly, the chamber having rigid chamber walls that are translucent or transparent to allow the patient's tongue to be viewed through the wall; and
a resilient hollow bulb directly connected to, and in fluid communication with, said chamber;
wherein said chamber has an open end through which a person's tongue can project,
wherein the device is configured such that the deforming and releasing of said bulb creates a reduced pressure within the chamber, thereby retaining a person's tongue within the chamber
wherein
the length of the chamber is 40-80 mm,
the width of the chamber opening is 20-60 mm,
the external width of the device in the region of the chamber is 37 to 51 mm at its widest point and
the external height of the device is 7 to 26 mm at its point of maximum height.

2. A device according to claim 1, wherein the chamber tapers in a direction from the open end towards the bulb.

3. A device according to claim 1, wherein the chamber and the bulb are connected by a connector, having an internal channel extending between the interior of the bulb and the chamber.

4. A device according to claim 3, wherein the outer surface of the connecting section has ribs of thicker material provided along its length.

5. A device according to claim 1, wherein the internal volume of the bulb is such that, in operation, it can maintain a sufficient negative pressure to retain a person's tongue within the chamber.

6. A device according to claim 1, wherein the device is 70 to 120 mm in length.

7. A device according to claim 1, wherein the bulb and the chamber wall are formed as two separate parts that may be joined together.

8. A device according to claim 1, which has been molded in a single piece.

9. A device as claimed in claim 1, which includes a passageway outside the chamber that is configured to allow air to enter the patient's mouth when the device is fitted.

10. A device as claimed in of claim 1, wherein the chamber wall includes an indention that, when the device is fitted to a patient, provides a passageway outside the chamber that allows air to enter the patient's mouth.

11. A device as claimed in claim 1, wherein the chamber as a length of 40-80 mm.

12. A method of clearing and/or holding open the airway of a person, wherein the method comprises holding the tongue of the person, using the device of claim 1.

13. A method according to claim 12, wherein the method comprises a first person:
compressing the bulb of the device according to claim 1,
engaging the tongue of a second person with the device,
releasing the bulb, to draw the tongue of the second person into the chamber of the device, optionally from the back of the throat.
